SEQUENCE LISTING (25 sequences)

SEQ ID NO: 1 - protein, 668 aa, unidentified
  REGION 1..668: note = Amino acid sequence encoded by CP77 gene
  source 1..668: note = Poxviridae
MFDYLENEEVALDELKQMLRDRDPNDTRNQFKNNALHAYLFNEHCNNVEVVKLLLDSGTNPLHKNWRQLTPLGEYTNSRHGKVNKDIAMVLLEATGYSNINDFNIFTYMKSKNVDIDLIKVLVEHGFDFSVKCEKHHSVIENYVMTDDPVPEIIDLFIENGCSVIYEDEDDEYGYAYEEYHSQNDDYQPRNCGTVLHLYIISHLYSESDSRSCVNPEVVKCLINHGINPSSIDKNYCTALQYYIKSSHIDIDIVKLLMKGIDNTAYSYIDDLTCCTRGIMADYLNSDYRYNKDVDLDLVKLFLENGKPHGIMCSIVPLWRNDKETISLILKTMNSDVLQHILIEYITFSDIDISLVEYMLEYGAVVNKEAIHGYFKNINIDSYTMKYLLKKEGGDAVNHLDDGEIPIGHLCKSNYGRYNFYTDTYRQGFRDMSYACPILSTINICLPYLKDINMIDKRGETLLHKAVRYNKQSLVSLLLESGSDVNIRSNNGYTCIAIAINESRNIELLNMLLCHKPTLDCVIDSLREISNIVDNAYAIKQCIRYAMIIDDCISSKIPESISKHYNDYIDICNQELNEMKKIIVGGNTMFSLIFTDHGAKIIHRYANNPELRAYYESKQNKIYVEVYDIISNAIVKHNKIHKNIESVDDNTYISNLPYTIKYKIFEQQ

SEQ ID NO: 2 - DNA, 2007 bp, unidentified
  misc_feature 1..2007: note = CP77 gene
  source 1..2007: mol_type = unassigned DNA; note = Poxviridae
atgttcgattacctggagaacgaagaagtggccctggacgagctgaagcagatgctgcgggatagggaccccaacgacaccagaaaccagtttaagaacaatgccctgcacgcctacctgttcaacgagcactgcaacaacgtggaagtggtcaagctgctgctggacagcggcaccaatcctctgcacaagaattggcggcagctgacccctctgggcgagtataccaacagccggcacggcaaagtgaacaaggatatcgccatggtgctgctcgaggccaccggctactccaacatcaacgacttcaacatctttacctacatgaagtccaagaacgtggacatcgacctgatcaaggtgctggtggaacacggcttcgacttcagcgtgaagtgcgagaagcaccacagcgtgatcgagaactacgtgatgaccgacgatcccgtgcctgagatcatcgatctgttcatcgagaacggctgctccgtgatctacgaagatgaggatgacgagtacggctacgcctacgaggaataccacagccagaacgacgattaccagcctcggaactgcggcaccgtgctgcacctgtacatcatcagccatctgtactccgagagcgacagcagatcctgcgtgaaccctgaggtggtcaagtgcctgatcaaccacggcatcaaccccagcagcatcgacaagaactactgcacagccctgcagtactacatcaagagcagccacattgacatcgatatcgtgaaactgctgatgaaggggatcgacaacaccgcctactcctacatcgacgacctgacctgctgcaccagaggcatcatggccgattacctgaacagcgactaccggtacaacaaggacgtggacctggacctcgtgaagctgttcctggagaacggcaagccccatggcatcatgtgcagcatcgtgcccctgtggcggaacgacaaagagacaatcagcctgatcctcaagaccatgaacagcgacgtgctccagcacatcctgatcgagtacatcaccttctccgacatcgacatcagcctggtcgagtatatgctggagtacggcgccgtggtcaacaaggaagccatccacggctacttcaagaatatcaatatcgacagctataccatgaagtacctgctgaagaaagaaggcggcgacgccgtcaaccacctggatgacggcgaaatccctatcggccacctgtgcaagagcaactacggcagatacaacttctacaccgacacctaccggcagggcttcagagacatgagctacgcctgtcctatcctgagcaccatcaacatctgcctgccttacctgaaggacatcaatatgatcgacaagcggggcgagacactgctgcacaaagccgtgcggtataacaagcagagcctggtgtccctgctcctggagagcggcagcgacgtgaacatcagaagcaacaacggctacacctgtatcgccatcgccattaatgagagccggaacatcgagctgctgaacatgctgctgtgccacaagcctacactggactgcgtgatcgacagcctgcgcgagatctccaatatcgtggacaacgcctacgccatcaagcagtgcatcagatacgccatgatcatcgacgattgcatcagcagcaagatccccgagagcatcagcaagcactacaacgattacattgacatctgcaatcaagagctgaacgaaatgaagaagatcatcgtcggcggcaacaccatgttctccctgatcttcacagatcacggcgctaagatcatccaccgctacgccaacaatcccgagctgagagcctactacgagagcaagcagaacaagatctacgtcgaggtgtacgacatcatcagcaacgccattgtgaagcacaacaaaatccacaagaacattgagagcgtggacgacaacacatatatcagcaatctgccttacacgatcaagtacaagatctttgaacagcagtga

SEQ ID NO: 3 - protein, 284 aa, unidentified
  REGION 1..284: note = Amino acid sequence encoded by K1L gene
  source 1..284: note = Poxviridae
MDLSRINTWKSKQLKSFLSSKDAFKADVHGHSALYYAIADNNVRLVCTLLNAGALKNLLENEFPLHQAATLEDTKIVKILLFSGLDDSQFDDKGNTALYYAVDSGNMQTVKLFVKKNWRLMFYGKTGWKTSFYHAVMLNDVSIVSYFLSEIPSTFDLAILLSCIHITIKNGHVDMMILLLDYMTSTNTNNSLLFIPDIKLAIDNKDIEMLQALFKYDINIYSANLENVLLDDAEIAKMIIEKHVEYKSDSYTKDLDIVKNNKLDEIISKNKELRLMYVNCVKKN

SEQ ID NO: 4 - DNA, 855 bp, unidentified
  misc_feature 1..855: note = K1L gene
  source 1..855: mol_type = unassigned DNA; note = Poxviridae
atggacctgagccggatcaacacctggaagtccaagcagctgaagtccttcctgagcagcaaggacgccttcaaggccgatgtgcacggacacagcgccctgtactatgccattgccgacaacaacgtgcggctcgtgtgcacccttctgaatgccggcgctctgaagaacctgctggaaaacgagttccctctgcaccaggccgccacactggaagataccaagatcgtgaagattctgctgttcagcggcctggacgacagccagttcgacgacaagggaaacaccgctctgtactacgccgtggacagcggcaatatgcagaccgtgaagctgttcgtgaagaaaaactggcggctgatgttctacggcaagaccggatggaaaaccagcttctaccacgccgtgatgctgaacgatgtgtctatcgtgtcctacttcctgtctgagatccccagcaccttcgacctggccatcctgctgagctgcatccacatcaccatcaagaacggccacgtggacatgatgatcctgctgctggactacatgaccagcaccaacaccaacaacagcctgctgtttatccccgacatcaagctggccatcgacaacaaggacatcgagatgctgcaggccctgtttaagtacgacatcaacatctacagcgccaacctcgagaacgtcctgctggacgatgccgagatcgccaagatgatcattgagaagcacgtcgagtacaagagcgacagctacaccaaggacctggacattgtgaagaacaacaagctggacgagatcatcagcaagaacaaagaactgcggcttatgtacgtgaactgcgtgaaaaagaactga

SEQ ID NO: 5 - protein, 906 aa, synthetic construct
  REGION 1..906: note = Amino acid sequence encoded by mRFP1-CP77 gene
MASSEDVIKEFMRFKVRMEGSVNGHEFEIEGEGEGRPYEGTQTAKLKVTKGGPLPFAWDILSPQFQYGSKAYVKHPADIPDYLKLSFPEGFKWERVMNFEDGGVVTVTQDSSLQDGEFIYKVKLRGTNFPSDGPVMQKKTMGWEASTERMYPEDGALKGEIKMRLKLKDGGHYDAEVKTTYMAKKPVQLPGAYKTDIKLDITSHNEDYTIVEQYERAEGRHSTGAGGGSGGGGSGGGGSFDYLENEEVALDELKQMLRDRDPNDTRNQFKNNALHAYLFNEHCNNVEVVKLLLDSGTNPLHKNWRQLTPLGEYTNSRHGKVNKDIAMVLLEATGYSNINDFNIFTYMKSKNVDIDLIKVLVEHGFDFSVKCEKHHSVIENYVMTDDPVPEIIDLFIENGCSVIYEDEDDEYGYAYEEYHSQNDDYQPRNCGTVLHLYIISHLYSESDSRSCVNPEVVKCLINHGINPSSIDKNYCTALQYYIKSSHIDIDIVKLLMKGIDNTAYSYIDDLTCCTRGIMADYLNSDYRYNKDVDLDLVKLFLENGKPHGIMCSIVPLWRNDKETISLILKTMNSDVLQHILIEYITFSDIDISLVEYMLEYGAVVNKEAIHGYFKNINIDSYTMKYLLKKEGGDAVNHLDDGEIPIGHLCKSNYGRYNFYTDTYRQGFRDMSYACPILSTINICLPYLKDINMIDKRGETLLHKAVRYNKQSLVSLLLESGSDVNIRSNNGYTCIAIAINESRNIELLNMLLCHKPTLDCVIDSLREISNIVDNAYAIKQCIRYAMIIDDCISSKIPESISKHYNDYIDICNQELNEMKKIIVGGNTMFSLIFTDHGAKIIHRYANNPELRAYYESKQNKIYVEVYDIISNAIVKHNKIHKNIESVDDNTYISNLPYTIKYKIFEQQ

SEQ ID NO: 6 - DNA, 2721 bp, synthetic construct
  misc_feature 1..2721: note = mRFP1-CP77 gene
  source 1..2721: mol_type = other DNA
atggccagcagcgaggacgtgatcaaagaattcatgcggttcaaagtgcggatggaaggcagcgtgaacggccacgagtttgagatcgaaggcgaaggcgagggcagaccttacgagggaacacagaccgccaagctgaaagtgaccaaaggcggccctctgccttttgcctgggacattctgagccctcagttccagtacggcagcaaggcctacgtgaagcaccctgccgacattcccgactacctgaagctgagcttccccgagggcttcaagtgggagagagtgatgaacttcgaggacggcggcgtggtcaccgtgactcaagatagctctctgcaggacggcgagttcatctacaaagtgaagctgcggggcaccaactttccctctgatggccccgtgatgcagaaaaagaccatgggctgggaagccagcaccgagagaatgtaccctgaagatggcgccctgaagggcgagatcaagatgcggctgaaactgaaggatggcggccactacgacgccgaagtgaaaaccacctacatggccaagaaacccgtgcagctgcctggcgcctacaagaccgatatcaagctggacatcaccagccacaacgaggactacaccatcgtggaacagtacgagagagccgaaggcagacactctacaggtgctggcggaggatctggtggtggtggatctggcggcggaggcagcttcgattacctggagaacgaagaagtggccctggacgagctgaagcagatgctgcgggatagggaccccaacgacaccagaaaccagtttaagaacaatgccctgcacgcctacctgttcaacgagcactgcaacaacgtggaagtggtcaagctgctgctggacagcggcaccaatcctctgcacaagaattggcggcagctgacccctctgggcgagtataccaacagccggcacggcaaagtgaacaaggatatcgccatggtgctgctcgaggccaccggctactccaacatcaacgacttcaacatctttacctacatgaagtccaagaacgtggacatcgacctgatcaaggtgctggtggaacacggcttcgacttcagcgtgaagtgcgagaagcaccacagcgtgatcgagaactacgtgatgaccgacgatcccgtgcctgagatcatcgatctgttcatcgagaacggctgctccgtgatctacgaagatgaggatgacgagtacggctacgcctacgaggaataccacagccagaacgacgattaccagcctcggaactgcggcaccgtgctgcacctgtacatcatcagccatctgtactccgagagcgacagcagatcctgcgtgaaccctgaggtggtcaagtgcctgatcaaccacggcatcaaccccagcagcatcgacaagaactactgcacagccctgcagtactacatcaagagcagccacattgacatcgatatcgtgaaactgctgatgaaggggatcgacaacaccgcctactcctacatcgacgacctgacctgctgcaccagaggcatcatggccgattacctgaacagcgactaccggtacaacaaggacgtggacctggacctcgtgaagctgttcctggagaacggcaagccccatggcatcatgtgcagcatcgtgcccctgtggcggaacgacaaagagacaatcagcctgatcctcaagaccatgaacagcgacgtgctccagcacatcctgatcgagtacatcaccttctccgacatcgacatcagcctggtcgagtatatgctggagtacggcgccgtggtcaacaaggaagccatccacggctacttcaagaatatcaatatcgacagctataccatgaagtacctgctgaagaaagaaggcggcgacgccgtcaaccacctggatgacggcgaaatccctatcggccacctgtgcaagagcaactacggcagatacaacttctacaccgacacctaccggcagggcttcagagacatgagctacgcctgtcctatcctgagcaccatcaacatctgcctgccttacctgaaggacatcaatatgatcgacaagcggggcgagacactgctgcacaaagccgtgcggtataacaagcagagcctggtgtccctgctcctggagagcggcagcgacgtgaacatcagaagcaacaacggctacacctgtatcgccatcgccattaatgagagccggaacatcgagctgctgaacatgctgctgtgccacaagcctacactggactgcgtgatcgacagcctgcgcgagatctccaatatcgtggacaacgcctacgccatcaagcagtgcatcagatacgccatgatcatcgacgattgcatcagcagcaagatccccgagagcatcagcaagcactacaacgattacattgacatctgcaatcaagagctgaacgaaatgaagaagatcatcgtcggcggcaacaccatgttctccctgatcttcacagatcacggcgctaagatcatccaccgctacgccaacaatcccgagctgagagcctactacgagagcaagcagaacaagatctacgtcgaggtgtacgacatcatcagcaacgccattgtgaagcacaacaaaatccacaagaacattgagagcgtggacgacaacacatatatcagcaatctgccttacacgatcaagtacaagatctttgaacagcagtga

SEQ ID NO: 7 - protein, 301 aa, synthetic construct
  REGION 1..301: note = Amino acid sequence encoded by K1L-V5 gene
MDLSRINTWKSKQLKSFLSSKDAFKADVHGHSALYYAIADNNVRLVCTLLNAGALKNLLENEFPLHQAATLEDTKIVKILLFSGLDDSQFDDKGNTALYYAVDSGNMQTVKLFVKKNWRLMFYGKTGWKTSFYHAVMLNDVSIVSYFLSEIPSTFDLAILLSCIHITIKNGHVDMMILLLDYMTSTNTNNSLLFIPDIKLAIDNKDIEMLQALFKYDINIYSANLENVLLDDAEIAKMIIEKHVEYKSDSYTKDLDIVKNNKLDEIISKNKELRLMYVNCVKKNGGSGKPIPNPLLGLDST

SEQ ID NO: 8 - DNA, 906 bp, synthetic construct
  misc_feature 1..906: note = K1L-V5 gene
  source 1..906: mol_type = other DNA
atggacctgagccggatcaacacctggaagtccaagcagctgaagtccttcctgagcagcaaggacgccttcaaggccgatgtgcacggacacagcgccctgtactatgccattgccgacaacaacgtgcggctcgtgtgcacccttctgaatgccggcgctctgaagaacctgctggaaaacgagttccctctgcaccaggccgccacactggaagataccaagatcgtgaagattctgctgttcagcggcctggacgacagccagttcgacgacaagggaaacaccgctctgtactacgccgtggacagcggcaatatgcagaccgtgaagctgttcgtgaagaaaaactggcggctgatgttctacggcaagaccggatggaaaaccagcttctaccacgccgtgatgctgaacgatgtgtctatcgtgtcctacttcctgtctgagatccccagcaccttcgacctggccatcctgctgagctgcatccacatcaccatcaagaacggccacgtggacatgatgatcctgctgctggactacatgaccagcaccaacaccaacaacagcctgctgtttatccccgacatcaagctggccatcgacaacaaggacatcgagatgctgcaggccctgtttaagtacgacatcaacatctacagcgccaacctcgagaacgtcctgctggacgatgccgagatcgccaagatgatcattgagaagcacgtcgagtacaagagcgacagctacaccaaggacctggacattgtgaagaacaacaagctggacgagatcatcagcaagaacaaagaactgcggcttatgtacgtgaactgcgtgaaaaagaacggcggcagcggcaagcccattcctaatccactgctgggcctcgacagcacctga

SEQ ID NO: 9 - protein, 356 aa, synthetic construct
  REGION 1..356: note = Amino acid sequence encoded by SPI-1 gene
MGGSDIFKELILKHTDENVLISPVSILSTLSILNHGAAGSTAEQLSKYIENMNENTPDDNNDMDVDIPYCATLATANKIYGSDSIEFHASFLQKIKDDFQTVNFNNANQTKELINEWVKTMTNGKINSLLTSPLSINTRMTVVSAVHFKAMWKYPFSKHLTYTDKFYISKNIVTSVDMMVSTENNLQYVHINELFGGFSIIDIPYEGNSSMVIILPDDIEGIYNIEKNITDEKFKKWCGMLSTKSIDLYMPKFKVEMTEPYNLVPILENLGLTNIFGYYADFSKMCNETITVEKFLHTTFIDVNEEYTEASAVTGVFMTNFSMVYRTKVYINHPFMYMIKDNTGRILFIGKYCYPQ

SEQ ID NO: 10 - DNA, 1071 bp, synthetic construct
  misc_feature 1..1071: note = SPI-1 gene
  source 1..1071: mol_type = other DNA
atgggaggcagcgacatcttcaaagagctgatcctgaagcacaccgacgagaacgtgctgatctcccctgtgtccatcctgagcaccctgtctatcctgaatcacggcgctgccggatctacagccgagcagctgagcaagtacatcgagaacatgaacgagaacaccccggacgacaacaacgatatggacgtggacatcccctactgcgccacactggccacagccaacaagatctacggcagcgactccatcgagttccacgccagctttctccagaagatcaaggacgacttccagaccgtgaacttcaacaacgccaaccagaccaaagaactgatcaacgagtgggtcaagaccatgaccaacggcaagatcaacagcctgctgacaagccctctgagcatcaacacccggatgaccgtggtgtccgccgtgcactttaaggccatgtggaagtaccccttcagcaagcacctgacctacaccgacaagttctacatcagcaagaacatcgtgaccagcgtggacatgatggtgtccaccgagaacaacctgcagtacgtgcacatcaacgagctgttcggcggcttcagcatcatcgacatcccttacgagggcaacagcagcatggtcatcatcctgcctgacgatatcgagggcatctacaatatcgagaagaacatcacggacgagaagttcaaaaagtggtgcggcatgctgagcaccaagagcatcgacctgtacatgcccaagttcaaggtggaaatgaccgagccttacaacctggtgcctatcctggaaaacctgggcctgaccaacatcttcggctactacgccgacttctccaagatgtgcaacgagacaattaccgtggaaaagttcctgcacaccaccttcatcgacgtgaacgaagagtacaccgaggcctctgctgtgaccggcgtgttcatgaccaatttcagcatggtgtaccggaccaaggtgtacatcaatcacccctttatgtacatgatcaaggataacaccggccggatcctgttcatcggcaagtactgctaccctcagtaa

SEQ ID NO: 11 - protein, 634 aa, unidentified
  REGION 1..634: note = Amino acid sequence encoded by C9L gene
  source 1..634: note = Poxviridae
MVNDKILYDSCKTFNIDASSAQSLIESGANPLYEYDGETPLKAYVTKKNNNIKNDVVILLLSSVDYKNINDFDIFEYLCSDNIDIDLLKLLISKGIEINSIKNGINIVEKYATTSNPNVDVFKLLLDKGIPTCSNIQYGYKIKIEQIRRAGEYYNWDDELDDYDYDYTTDYDDRMGKTVLYYYIITRSQDGYATSLDVINYLISHKKEMRYYTYREHTTLYYYLDKCDIKREIFDALFDSNYSGHELMNILSNYLRKQFRKKNHKIDNYIVDQLLFDRDTFYILELCNSLRNNILISTILKRYTDSIQDLLLEYVSYHTVYINVIKCMIDEGATLYRFKHINKYFQKFGNRDPKVVEYILKNGNLVVDNDNDDNLINIMPLFPTFSMRELDVLSILKLCKPYIDDINKIDKHGCSILYHCIKSHSVSLVEWLIDNGADINIITKYGFTCITICVILADKYIPEIAELYIKILEIILSKLPTIECIKKTVDYLDDHRYLFIGGNNKSLLKICIKYFILVDYKYTCSMYPSYIEFITDCEKEIADMRQIKINGTDMLTVMYMLNKPTKKRYVNNPIFTDWANKQYKFYNQIIYNANKLIEQSKKIDDMIEEVSIDDNRLSTLPLEIRHLIFSYAFL

SEQ ID NO: 12 - DNA, 1905 bp, unidentified
  misc_feature 1..1905: note = C9L gene
  source 1..1905: mol_type = unassigned DNA; note = Poxviridae
atggttaacgataagatactctatgatagttgtaaaacatttaacatcgatgccagcagtgcacaatcattgatagaaagtggtgcaaatccattatatgagtatgatggtgaaactccattaaaggcatacgttaccaagaaaaataataatatcaaaaacgatgttgtgattttgttattgtcgtcagtcgactataaaaatatcaatgattttgatatattcgaatatctatgttctgataacatcgatatagacttattgaaattactaatttcgaaaggtatagaaataaatagtatcaaaaatggtattaatattgtagagaaatacgctacaacatcaaatcccaatgtagatgtgtttaaactattattggataaaggaatacctacatgtagcaacatacagtatggatacaagatcaaaatagaacagattagacgtgctggtgaatattataattgggatgatgaattagacgattacgattacgactacaccactgattatgatgatagaatgggtaaaacagttctctattattatattattactaggtcacaagatggttatgctacatctttggacgtgataaactatttaatttcacacaaaaaagagatgcgttattatacttatcgtgaacataccacactctattattatcttgacaaatgcgatattaaacgggaaatatttgacgcgttattcgatagtaactatagtggtcatgaactaatgaatattctatctaactatttacgtaaacagtttaggaagaaaaatcacaaaatcgataattatatagttgatcaactattattcgaccgtgatacgttttatattttagaattgtgtaatagtttacgtaataatatcctaatatccacaattcttaaaagatatacagattctatacaagatctattgttagaatatgtatcttatcatacagtatacatcaatgttattaaatgtatgattgatgaaggagctacattatatagatttaagcatataaataaatattttcaaaaatttggcaatagagatcctaaagttgtcgagtatattttaaaaaatggaaacttagttgtagataatgacaatgatgataacctaataaatattatgccattattccctaccttctctatgcgtgagttggatgtgttatcgatactaaaactttgtaagccgtatattgatgatataaacaaaatagataaacatggatgtagtatactttatcattgtattaagtcgcatagtgtcagcctagtagaatggttaatagataatggcgcagacattaatataataacaaaatatgggtttacatgtattactatttgtgttatactggcagataaatatatcccagaaatagcagaattatatattaagatattggaaattattctgagtaaattaccaaccatcgaatgtattaagaaaacagttgattacctagacgatcacaggtacttattcataggtggtaataataaatcgttactgaaaatatgtatcaagtacttcatattagtcgattataagtacacatgtagcatgtatccatcatatatagaatttataaccgactgcgaaaaagaaattgcggatatgcgtcaaattaaaataaatggtacggacatgcttacagtgatgtacatgttaaataaacctacaaagaaacgatatgttaataatccgatatttacagattgggctaataagcaatataagttttataatcaaataatatataatgctaataagttaatagaacaaagtaagaaaatagacgacatgatagaggaggtatccattgacgataatcgtttatcaacactaccgttagaaattagacatttgattttctcgtacgcgttcctataa

SEQ ID NO: 13 - DNA, 108 bp, synthetic construct
  misc_feature 1..108: note = PrH5m promoter
  source 1..108: mol_type = other DNA
taaaaattgaaaataaatacaaaggttcttgagggttgtgttaaattgaaagcgagaaataatcataaataatttcattatcgcgatatccgttaagtttgtatcgta

SEQ ID NO: 14 - DNA, 100 bp, synthetic construct
  misc_feature 1..100: note = Pr1328 promoter
  source 1..100: mol_type = other DNA
tatattattaagtgtggtgtttggtcgatgtaaaatttttgtcgataaaaattaaaaaataacttaatttattattgatctcgtgtgtacaaccgaaatc

SEQ ID NO: 15 - DNA, 124 bp, synthetic construct
  misc_feature 1..124: note = Pr13.5 promoter
  source 1..124: mol_type = other DNA
taaaaatagaaactataatcatataatagtgtaggttggtagtattgctcttgtgactagagactttagttaaggtactgtaaaaatagaaactataatcatataatagtgtaggttggtagta

SEQ ID NO: 16 - DNA, 20 bp, synthetic construct
  misc_feature 1..20: note = EGFP forward primer
  source 1..20: mol_type = other DNA
cagctcgtccatgccgagag

SEQ ID NO: 17 - DNA, 20 bp, synthetic construct
  misc_feature 1..20: note = EGFP reverse primer
  source 1..20: mol_type = other DNA
tgaagttcatctgcaccacc

SEQ ID NO: 18 - DNA, 23 bp, synthetic construct
  misc_feature 1..23: note = CP77 forward primer
  source 1..23: mol_type = other DNA
tgaaccctgaggtggtcaagtgc

SEQ ID NO: 19 - DNA, 22 bp, synthetic construct
  misc_feature 1..22: note = CP77 reverse primer
  source 1..22: mol_type = other DNA
cgatcaggatgtgctggagcac

SEQ ID NO: 20 - DNA, 19 bp, synthetic construct
  misc_feature 1..19: note = K1L forward primer
  source 1..19: mol_type = other DNA
cgatgtgcacggacacagc

SEQ ID NO: 21 - DNA, 22 bp, synthetic construct
  misc_feature 1..22: note = K1L reverse primer
  source 1..22: mol_type = other DNA
gctggtcatgtagtccagcagc

SEQ ID NO: 22 - DNA, 22 bp, synthetic construct
  misc_feature 1..22: note = SPI-1 forward primer
  source 1..22: mol_type = other DNA
cgatcaggatgtgctggagcac

SEQ ID NO: 23 - DNA, 22 bp, synthetic construct
  misc_feature 1..22: note = SPI-1 reverse primer
  source 1..22: mol_type = other DNA
gtacaccttggtccggtacacc

SEQ ID NO: 24 - DNA, 27 bp, synthetic construct
  misc_feature 1..27: note = C9L forward primer
  source 1..27: mol_type = other DNA
catgctacatgtgtacttataatcgac

SEQ ID NO: 25 - DNA, 29 bp, synthetic construct
  misc_feature 1..29: note = C9L reverse primer
  source 1..29: mol_type = other DNA
gccgtatattgatgatataaacaaaatag

(19) United States
(12) Patent Application Publication
(10) Pub. No.: US 2026/0078353 A1
(43) Pub. Date: Mar. 19, 2026

(54) Mammalian Cell Line for the Production of Modified Vaccinia Virus Ankara (MVA)

(71) Applicant: Bavarian Nordic A/S, Hellerup (DK)

(72) Inventors: Robin Steigerwald, Munich (DE); Zhiyong Xu, Munich (DE); Juergen Hausmann, Gundelfingen (DE)

(73) Assignee: Bavarian Nordic A/S, Hellerup (DK)

(21) Appl. No.: 18/879,789

(22) PCT Filed: Jun. 30, 2023
(86) PCT No.: PCT/EP2023/067987

§ 371 Date: Dec. 29, 2024

(30) Foreign Application Priority Data
Jun. 30, 2022 (EP) 22182381.8

(51) Int. Cl. C12N 7/02; A61K 39/00; A61K 39/12; C12N 5/071; C12N 5/10; C12N 7/04

(52) U.S. Cl. CPC C12N 7/025; A61K 39/12; C12N 5/0682; C12N 5/10; C12N 7/04; A61K 2039/5254; C12N 2510/02; C12N 2710/24134; C12N 2710/24152

(57) ABSTRACT

The present invention relates to a mammalian non-human cell line, specifically Chinese hamster ovary (CHO) cells, that is genetically modified to express poxvirus host range genes CP77, K1L and/or SPI-1 which are not expressed in MVA, and to the use of said cell line in the reproduction of MVA.

### TECHNICAL FIELD

The present invention relates to the field of viral vector-based vaccines. More particularly, the present invention relates to mammalian cell substrates for reproduction of Modified Vaccinia Virus Ankara (MVA). In particular, the present invention relates to a mammalian non-human cell line, specifically Chinese hamster ovary (CHO) cells, that is genetically modified to express poxvirus host range genes not expressed in MVA. The present invention furthermore relates to the generation of such cell lines and their use in the production of MVA-based vaccines. The present invention also relates to recombinant MVA expressing poxvirus host range genes not intrinsically expressed in MVA.

### BACKGROUND

Modified Vaccinia Virus Ankara (MVA) is a highly attenuated subspecies of vaccinia virus (genus Orthopoxvirus). Strain MVA-BN® (strain Bavarian Nordic) has been approved for use as a safe human vaccine against smallpox and monkeypox (1-3). Additionally, MVA is widely used as a safe viral vector for the generation of recombinant vaccines against infectious diseases and cancer (3-5).

MVA was derived by continuously passaging the ancestor Chorioallantois Vaccinia Virus Ankara (CVA) in chicken embryo fibroblasts (CEFs or CEF cells) for more than 500 times (3, 6). During passaging, multiple mutations and deletions accumulated in the viral genome. Compared to the parental CVA, MVA lacks six large genomic fragments and acquired multiple mutations and small deletions throughout its genome (7-9). As a result, MVA lost the ability to replicate in most mammalian cells and only a few cell lines are known to support productive viral replication. Thus, as compared to the parental CVA and other members of Orthopoxvirus, MVA is characterized by a distinct restriction of the virus' host range.

To date, the only cell substrate approved for MVA-based vaccine production are CEF cells (10, 11). Even though primary CEF cells are a well-established cell substrate for MVA production, their preparation from embryonated eggs is laborious and time-consuming. Moreover, the procedure is prone to contamination. All that is not ideal for a stable and efficient production process, particularly not for the manufacturing of vaccines on an industrial scale and under the strict requirements of Good Manufacturing Practice (GMP).

Thus, there is a need for alternative cell substrates for the reproduction of MVA.

U.S. Pat. No. 5,830,688 (dated 1998) describes the identification of a sequence involved in the multiplication of cowpox virus in Chinese hamster ovary (CHO) cells and which, when transferred into vaccinia virus (intrinsically incapable of multiplying in CHO cells) allows the multiplication of vaccinia in this cell line.

However, replication deficiency in most mammalian cell lines is an important safety feature of MVA in view of its use for human vaccines. Therefore, genetic modification such that MVA is rendered capable of replication in mammalian cells is not an option.

Rather, it has been considered to express one or more poxvirus host range genes that are able to support MVA replication in an otherwise non-susceptible cell line. If the appropriate host range genes were expressed, the modified cells should gain the ability to help replicate MVA to acceptable titers.

The genetic basis of MVA host range restriction is not well understood and for most cell lines, the responsible poxvirus genes remain largely unknown. At least, the six major genome deletions in MVA (related to CVA) are not alone responsible for the strong attenuation and highly restricted host range characteristic of MVA (12). This indicates that the host range restriction of MVA is most likely a cooperative effect involving not only the six major deletions, but also other smaller gene deletions and mutations present throughout the genome (12).

Among the well-defined host range genes of Orthopoxvirus, the cowpox virus (CPXV or CWPX virus) CP77 gene is known to promote vaccinia virus (VACV) and ectromelia virus replication in Chinese hamster ovary (CHO) and RK13 (from rabbit kidney) cells (13-17). A genetically engineered CHO cell line expressing CP77 and the VACV-derived D13L gene was generated for production of replication-defective recombinant VACV (18). The expression of CP77 gene was also shown to expand the host range of MVA by alleviating a block before late protein synthesis in CHO cells (19). However, CP77 alone may not be sufficient for high-titer MVA replication in otherwise non-permissive cells.

In addition to CP77, MVA lacks at least three other genes, namely K1L, SPI-1 (C12L) and C9L, which may also contribute to the host range of Orthopoxvirus in different cell lines (20-25). K1L is a VACV host range gene known to be involved in the negative regulation of the NF-κB signaling pathway and can restore replication of MVA in RK13 cells (21, 26). It has also been reported that K1L gene is responsible for VACV replication in RK13 cells, and CP77 can complement the effect of K1L gene in this cell line [18].

The host range function of serine protease inhibitor 1 (SPI-1) was first found in rabbitpox virus and has been shown to act as an apoptosis inhibitor (22, 27). A VACV SPI-1 deletion mutant cannot replicate efficiently in primary human keratinocytes or human lung carcinoma cells (A549) (28). Meanwhile, SPI-1 has been identified as a host range factor for MVA (29). Moreover, A549 cells co-expressing a recently identified host range gene, i.e., C16L/B22R, together with SPI-1 gene turned out to be permissive for MVA (30).

The vaccinia C9L gene has been shown to act as an inhibitor of the host immune response by antagonizing the action of interferons early in the viral replication cycle (23).

The expression of host ranges genes CP77, K1L, SPI-1, and C9L in MVA and other members of Orthopoxvirus are summarized in Table 1 below. Additionally, cell lines not supporting the replication of a host range gene deficient virus are listed in Table 1.

TABLE 1
Left: Poxvirus host range gene. Middle: Intrinsic expression
of host range gene in Orthopoxvirus (+/−). Right:
Culture cells not compensating for a deficiency in host range gene.

| | Orthopoxvirus | | | | | |
|---|---|---|---|---|---|---|
| Gene | MVA | CVA | COP | WR | CPXV | Culture cells |
| SPI-1 | − | + | + | + | + | A549 (human |
| | | | | | | keratinocytes) |
| K1L | − | + | + | + | + | RK13 (rabbit kidney |
| | | | | | | epithelium) |
| C9L | − | − | + | + | + | A549, IFNβ pre-treated |
| CP77 | − | − | − | − | + | CHO (Chinese hamster |
| | | | | | | ovary) RK13 |

Abbr.: COP = Vaccinia virus strain Copenhagen; WR = Vaccinia virus strain Western Reserve; CPXV = Cowpox virus; IFNβ = Interferon β.

### SUMMARY OF INVENTION

It is an object of the present invention to provide a cell substrate for reproducing MVA.

The object of the present invention is solved by the provision of a CHO cell expressing a combination of poxvirus host range genes not expressed by MVA. In particular, the invention is defined by the appended claims and by the following aspects and their embodiments.

In a first aspect, the invention provides a cell of a continuous cell line, the cell being genetically modified to express poxvirus host range genes CP77 and K1L.

In a particular aspect, the invention provides a cell of a continuous cell line, the cell being genetically modified to express poxvirus host range genes CP77, K1L and SPI-1.

In another aspect, the invention provides the use of a cell according to the invention for reproduction of MVA.

In yet another aspect, the invention provides the use of a cell according to the invention in the production of a vaccine comprising MVA.

In yet another aspect, the invention provides a vaccine comprising MVA, the MVA being prepared using a cell according to the invention.

In yet another aspect, the invention provides a method for generating a cell according to the invention, comprising the following steps:
(a) Preparing a nucleic acid suitable for gene transfer into a cell of a continuous cell line, the nucleic acid comprising:
   (i) poxvirus host range gene CP77 operably linked to a promoter; or
   (ii) poxvirus host range gene K1L operably linked to a promoter; or
   (iii) poxvirus host range genes CP77 and K1L, each operably linked to a promoter;
(b) Introducing nucleic acids (i) and (ii) obtained in step (a), or nucleic acid (iii) obtained in step (a), into the cell; and
(c) Selecting a cell population or clone expressing poxvirus host range genes CP77 and K1L.

In yet another aspect, the invention provides a method for generating a cell according to the invention, comprising the following steps:
(a) Preparing a nucleic acid suitable for gene transfer into a cell of a continuous cell line, the nucleic acid comprising:
   (i) poxvirus host range gene CP77 operably linked to a promoter; or
   (ii) poxvirus host range gene K1L operably linked to a promoter; or
   (iii) poxvirus host range gene SPI-1 operably linked to a promoter; or
   (iv) poxvirus host range genes CP77, K1L and SPI-1, each operably linked to a promoter;
(b) Introducing nucleic acids (i), (ii) and (iii) obtained in step (a), or nucleic acid (iv) obtained in step (a), into the cell; and
(c) Selecting a cell population or clone expressing poxvirus host range genes CP77, K1L and SPI-1.

In yet another aspect, the invention provides a cell generated by a method according to the invention.

In yet another aspect, the invention provides a MVA reproduced using a cell according to the invention.

In yet another aspect, the invention provides the use of poxvirus host range genes CP77 and K1L for rendering a culture cell capable of expressing CP77 and K1L.

In yet another aspect, the invention provides the use of poxvirus host range genes CP77, K1L and SPI-1 for rendering a cell capable of expressing CP77, K1L and SPI-1.

In yet another aspect, the invention provides a MVA comprising poxvirus host range genes CP77 and K1L,

In yet another aspect, the invention provides a MVA comprising poxvirus host range genes CP77, K1L and SPI-1.

### BRIEF DESCRIPTION OF DRAWINGS/FIGURES

FIG. 1 schematically illustrates the design of vaccinia virus (MVA or CVA) having inserted in its genome one or more host range genes.

C=CP77, K=K1L-V5, S=SPI-1, C9=C9L; mRFP1-CP77=mRFP1-CP77 fusion gene. Promoters: PrH5m, Pr1328, Pr13.5, pS. Insertion sites: 059-060 (CVA), 44L-45L (MVA), 88R-89L (MVA).

CVA-wt and MVA-wt were reconstituted from BAC clones each containing a bi-cistronic expression cassette under the control of the strong synthetic early/late pS promoter encoding the NPT II-IRES-EGFP cassette. CVA-C was generated by inserting the mRFP-CP77 gene into CVA-wt using homologous recombination. To generate MVA recombinants, host range genes of interest were inserted into MVA BAC clones using the A red recombination technology.

FIG. 2 shows the transcription of inserted host range genes by vaccinia virus grown in CEF cells (as analyzed by RT-PCR).

Upper row: Recombinant MVA or CVA as compared to the corresponding wildtype (MVA-wt and CVA-wt, respectively). RT=reverse transcriptase; “+”=sample treated with RT; “−”=control sample without RT treatment.

Left lane: Gene of interest and size of the related PCR product. Right lane: Size of bands in the 100 bp DNA ladder (New England Biolabs).

FIG. 3 shows microscopic pictures (red and green fluorescence, phase contrast) of CHO cells infected with MVA (wildtype wt or recombinant) or CVA (wildtype wt or recombinant). Pictures were taken 72 hours after infection with vaccinia virus and incubation of the cells. Upper row: MVA-wt and MVA recombinants, CVA-wt and CVA recombinant; presence (“+”) or absence (“−”) of EGFP (enhanced green fluorescence protein) gene and host range genes CP77 (coupled to mRFP1, mono red fluorescence protein), K1L-V5 and SPI-1.

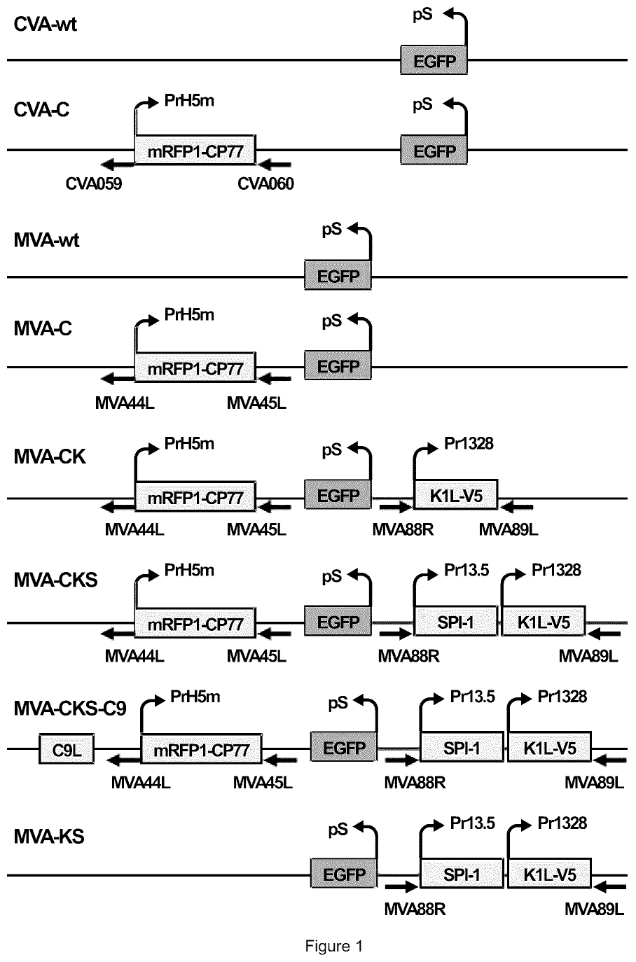

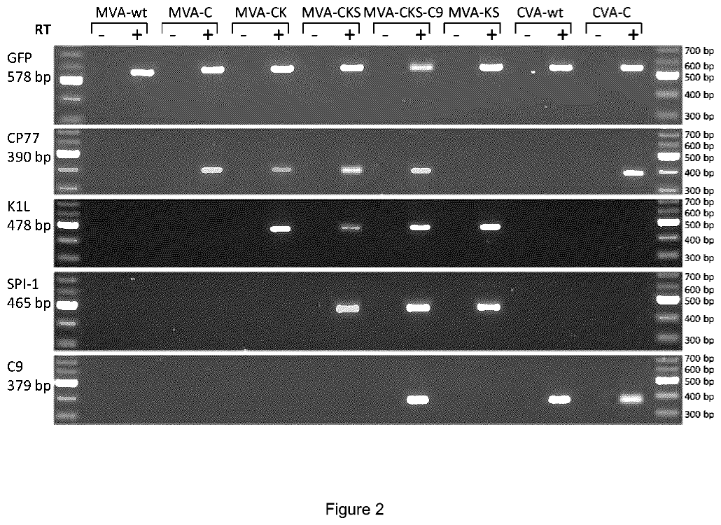

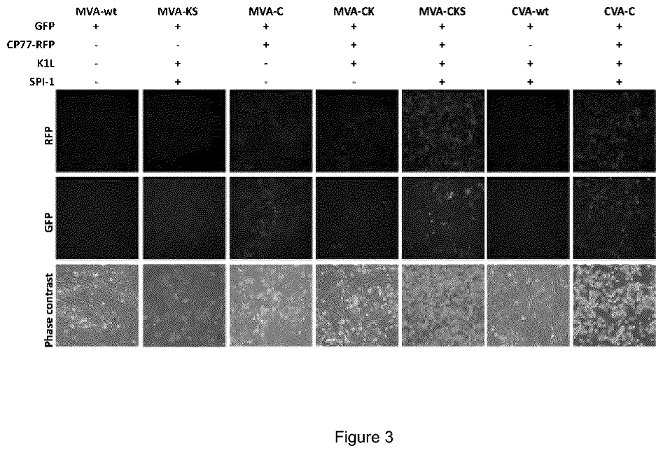

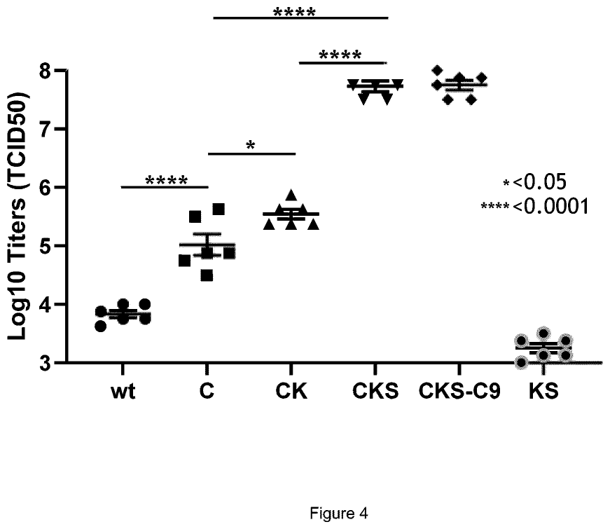

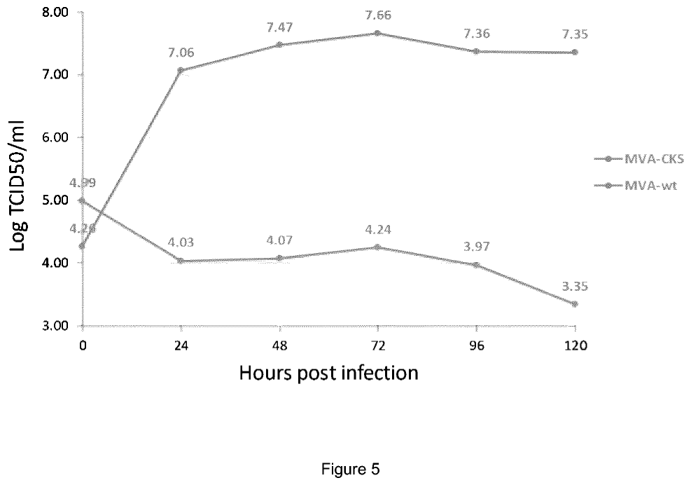

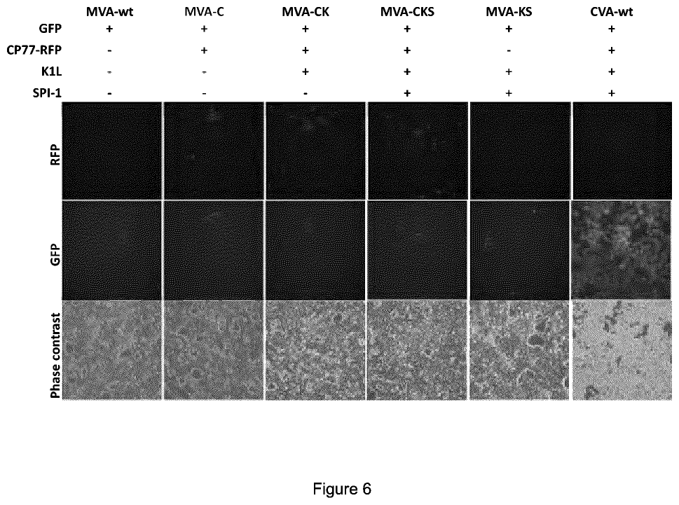

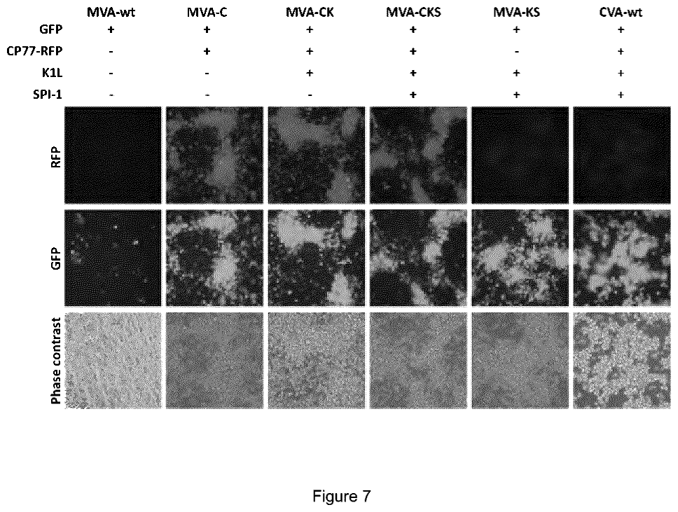

FIG. 4 shows the replication capacity of MVA (wildtype wt or recombinant) in CHO cells. Infected cells and supernatants were harvested 48 hours after infection with MVA and incubation of the cells. For abbreviations of host range genes inserted into MVA (x-axis) see legend to FIG. 1.

FIG. 5 shows the growth kinetics of MVA encoding host range genes CP77, K1L and SPI-1 (“MVA-CKS”) and wildtype (“MVA-wt”) in CHO cells.

FIG. 6 shows microscopic pictures (red and green fluorescence, phase contrast) of HEK293 cells infected with MVA (wildtype wt or recombinant) or CVA (wildtype wt) at 72 hours post infection.

FIG. 7 shows microscopic pictures (red and green fluorescence, phase contrast) of RK13 cells infected with MVA (wildtype wt or recombinant) or CVA (wildtype wt) at 72 hours post infection.

Brief Description of Sequences

| | |
|---|---|
| SEQ ID NO: 1 | depicts the amino acid sequence encoded by CP77 gene. |
| SEQ ID NO: 2 | depicts the nucleic acid sequence of CP77 gene. |
| SEQ ID NO: 3 | depicts the amino acid sequence encoded by K1L gene. |
| SEQ ID NO: 4 | depicts the nucleic acid sequence of K1L gene. |
| SEQ ID NO: 5 | depicts the amino acid sequence encoded by mRFP1-CP77 gene. |
| SEQ ID NO: 6 | depicts the nucleic acid sequence of mRFP1-CP77 gene. |
| SEQ ID NO: 7 | depicts the amino acid sequence encoded by K1L-V5 gene. |
| SEQ ID NO: 8 | depicts the nucleic acid sequence of K1L-V5 gene. |
| SEQ ID NO: 9 | depicts the amino acid sequence encoded by SPI-1 gene. |
| SEQ ID NO: 10 | depicts the nucleic acid sequence of SPI-1 gene. |
| SEQ ID NO: 11 | depicts the amino acid sequence encoded by C9L gene. |
| SEQ ID NO: 12 | depicts the nucleic acid sequence of C9L gene. |
| | Note: The nucleotide sequences of mRFP1-CP77, K1L-V5 and |
| | SPI-1 are codon modified for expression in human cells. The |
| | C9L gene was PCR-amplified from VACV-WR genomic DNA. |
| SEQ ID NO: 13 | depicts the nucleic acid sequence of PrH5m promoter. |
| SEQ ID NO: 14 | depicts the nucleic acid sequence of Pr1328 promoter. |
| SEQ ID NO: 15 | depicts the nucleic acid sequence of Pr13.5 promoter. |
| SEQ ID NO: 16 | depicts the nucleic acid sequence of a GFP forward primer. |
| SEQ ID NO: 17 | depicts the nucleic acid sequence of a GFP reverse primer. |
| SEQ ID NO: 18 | depicts the nucleic acid sequence of a CP77 forward primer. |
| SEQ ID NO: 19 | depicts the nucleic acid sequence of a CP77 reverse primer. |
| SEQ ID NO: 20 | depicts the nucleic acid sequence of a K1L forward primer. |
| SEQ ID NO: 21 | depicts the nucleic acid sequence of a K1L reverse primer. |
| SEQ ID NO: 22 | depicts the nucleic acid sequence of a SPI-1 forward primer. |
| SEQ ID NO: 23 | depicts the nucleic acid sequence of a SPI-1 reverse primer. |
| SEQ ID NO: 24 | depicts the nucleic acid sequence of a C9L forward primer. |
| SEQ ID NO: 25 | depicts the nucleic acid sequence of a C9L reverse primer. |

### DETAILED DESCRIPTION OF INVENTION

Herein, we report the expansion of MVA's host range by introducing poxviral host range genes into the virus' genome. Host range genes CP77, K1L, SPI-1, and C9L were sequentially inserted into MVA, and the MVA recombinants produced in each step were screened for their ability to infect and replicate in continuous cell lines.

Amongst the cell lines tested, only CHO and RK13 cells (both being not permissive for MVA) allowed replication of MVA recombinants expressing at least the CP77 gene.

As shown for CHO cells, insertion of K1L gene into the MVA recombinant already encoding CP77 increased the recombinant's ability to replicate, and the yet additional insertion of SPI-1 gene enhanced its replication capacity further. The latter step, i.e., the insertion of SPI-1 gene in addition to CP77 and K1L genes, resulted even in the most pronounced improvement in MVA's replication capacity within the stepwise host range gene insertion procedure. In contrast, the insertion of C9L gene in addition to CP77, K1L and SPI-1 genes did not lead to further improvement.

Notably, the MVA recombinant expressing CP77, K1L and SPI-1 genes replicated in CHO cells to titers comparable to that of wildtype MVA grown in its standard substrate, i.e., primary CEF cells. On this basis, CHO cells genetically modified to co-express poxviral host range genes CP77, K1L and optionally SPI-1 were considered excellent substrates for MVA reproduction.

CHO is a continuous non-human cell line widely used for manufacture of biologics in the biopharmaceutical industry. They can be grown in chemically defined growth media as suspension cultures to high cell densities in industrial scale bioreactors. Therefore, the possibility to use CHO cells as an alternative to primary CEF cells for reproducing MVA provides a significant advance in MVA-based vaccine production.

### Definitions

It must be noted that, as used herein, the singular forms “a”, “an”, and “the”, include plural references unless the context clearly indicates otherwise. Thus, for example, reference to “a nucleic acid sequence” includes one or more nucleic acid sequences.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the appended claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used in the context of an aspect or embodiment in the description of the present invention the term “comprising” can be amended and thus replaced with the term “containing” or “including” or when used herein with the term “having.” Similarly, any of the afore mentioned terms (comprising, containing, including, having), whenever used in the context of an aspect or embodiment in the description of the present invention include, by virtue, the terms “consisting of” or “consisting essentially of,” which each denotes specific legal meaning depending on jurisdiction.

When used herein “consisting of” excludes any element, step, or ingredient not specified in the claim element. When used herein, “consisting essentially of” does not exclude materials or steps that do not materially affect the basic and novel characteristics of the claim.

The term “MVA recombinant” as used herein refers to MVA having inserted one or more poxvirus host range genes not intrinsically expressed in native MVA and additionally an EGFP gene. Similarly, “CVA recombinant” refers to CVA having inserted a poxvirus host range gene not expressed in native CVA and additionally an EGFP gene.

The term “native MVA” or “native CVA” as used herein refers to MVA and CVA, respectively, that is not genetically modified. The term “not intrinsically expressed” refers to a gene that is not expressed or not contained in native MVA or CVA.

The term “wildtype MVA” (MVA-wt) or “wildtype CVA” (CVA-wt) refers to MVA and CVA, respectively, containing an EGFP gene and being used as controls for the recombinant MVA or CVA, respectively.

A virus' “host range” is the range of cell types, the virus is capable of infecting. The term “host range genes” refers to the genetic basis of the virus' host range.

The term “primary cell culture” or “primary culture cell” refers to an early stage of a cell culture after cells have been isolated from tissue. The opposite is continuous or immortalized cell lines. The term “continuous cell line” refers to cells that can be serially propagated in culture.

The term “permissive cell line” refers to cells that allow a virus to replicate. The term “permissiveness” refers to the capability of the cells to allow the virus replication.

The term “cell substrate” refers to a cell that allows reproduction of a virus, here of MVA.

The term “reproduction” refers to the replication or propagation of MVA.

The wording “cell being genetically modified to express” as used herein means that without and prior to the modification the cell is not capable of expressing a gene

### Abbreviations

BAC bacterial artificial chromosome
CEF chicken embryo fibroblast
CHO cell Chinese hamster ovary cell
CPXV, CWPX cowpox virus
CVA chorioallantois vaccinia virus Ankara
CVA-C CVA-wt encoding mRFP1-CP77
CVA-wt CVA wildtype encoding EGFP
EGFP enhanced green fluorescent protein
mRFP1 mono red fluorescent protein
MVA Modified Vaccinia Virus Ankara
MVA-BN® MVA-Bavarian Nordic, a proprietary strain of Bavarian Nordic
MVA-C MVA-wt encoding mRFP1-CP77
MVA-CK MVA-wt encoding mRFP1-CP77 and K1L-V5
MVA-CKS MVA-wt encoding mRFP1-CP77, K1L-V5 and SPI-1
MVA-CKS-C9 MVA-wt encoding mRFP1-CP77, K1L-V5, SPI-1, and C9L
MVA-KS MVA-wt encoding K1L and SPI-1
MVA-wt MVA reconstituted from a BAC clone of MVA-BN® encoding EGFP
ORF open reading frame
RT-PCT reverse transcription polymerase chain reaction
VACV vaccinia virus
VACV-WR vaccinia virus strain Western Reserve

### EMBODIMENTS

In one aspect, the invention provides a cell of a continuous cell line capable of expressing poxvirus host range genes CP77 and K1L.

In one aspect, the invention provides a cell of a continuous cell line that is genetically modified to express poxvirus host range genes CP77 and K1L.

In one embodiment, the genome of the cell comprises a nucleotide sequence encoding an amino acid sequence of SEQ ID NO: 1 and a nucleotide sequence encoding an amino acid sequence of SEQ ID NO: 3.

In one embodiment, the genome of the cell comprises a nucleotide sequence of SEQ ID NO: 2 and a nucleotide sequence of SEQ ID NO: 4.

In a particular aspect, the invention provides a cell of a continuous cell line capable of expressing poxvirus host range genes CP77, K1L and SPI-1.

In a particular aspect, the invention provides a cell of a continuous cell line that is genetically modified to express poxvirus host range genes CP77, K1L and SPI-1.

In one embodiment, the genome of the cell comprises a nucleotide sequence encoding an amino acid sequence of SEQ ID NO: 1, a nucleotide sequence encoding an amino acid sequence of SEQ ID NO: 3, and a nucleotide sequence encoding an amino acid sequence of SEQ ID NO: 9.

In one embodiment, the genome of the cell comprises a nucleotide sequence of SEQ ID NO: 2, a nucleotide sequence of SEQ ID NO: 4, and a nucleotide sequence of SEQ ID NO: 10.

In one embodiment, the cell is not capable of expressing a host range gene without and prior to genetic modification.

In one embodiment, the cell is infected with MVA.

In one embodiment of all aspects, the cell is a culture cell or a non-primary cell.

In one embodiment of all aspects, the cell is not a CEF, DF-1 or quail cell, or is a non-avian cell.

In one embodiment of all aspects, the cell line is mammalian cell line, preferably a non-human mammalian cell line.

In one embodiment of all aspects, the cell is a CHO cell.

In another aspect, the invention provides the use of a cell according to the invention for the reproduction of MVA.

In another aspect, the invention provides the use of a cell according to the invention in the production of a vaccine comprising MVA.

In yet another aspect, the invention provides a vaccine comprising MVA, the MVA or the vaccine being prepared using a cell according to the invention.

In yet another aspect, the invention provides a method for generating a cell according to the invention, comprising the following steps:
(a) Preparing a nucleic acid suitable for gene transfer into a cell of a continuous cell line, the nucleic acid comprising:
   (i) poxvirus host range gene CP77 operably linked to a promoter; or
   (ii) poxvirus host range gene K1L operably linked to a promoter; or
   (iii) poxvirus host range genes CP77 and K1L, each operably linked to a promoter;
(b) Introducing nucleic acids (i) and (ii) obtained in step (a), or nucleic acid (iii) obtained in step (a), into the cell; and
(c) Selecting a cell population or clone expressing poxvirus host range genes CP77 and K1L.

In yet another aspect, the invention provides a method for generating a cell according to the invention, comprising the following steps:
(a) Preparing a nucleic acid suitable for gene transfer into a cell of a continuous cell line, the nucleic acid comprising:
   (i) poxvirus host range gene CP77 operably linked to a promoter; or
   (ii) poxvirus host range gene K1L operably linked to a promoter; or
   (iii) poxvirus host range gene SPI-1 operably linked to a promoter; or
   (iv) poxvirus host range genes CP77, K1L and SPI-1, each operably linked to a promoter;
(b) Introducing nucleic acids (i), (ii) and (iii) obtained in step (a), or nucleic acid (iv) obtained in step (a), into the cell; and
(c) Selecting a cell population or clone expressing poxvirus host range genes CP77, K1L and SPI-1.

In one embodiment of the method, the nucleic acid suitable for gene transfer is a vector or a plasmid.

In one embodiment of the method, the promoter operably linked to a host range gene is one that is active in the cell into which the nucleic acid obtained in step (a) is transferred.

In one embodiment of the method, the promoter operably linked to a host range gene is a eukaryotic promoter.

In one embodiment of the method, the nucleic acid obtained in step (a) comprises a polyadenylation signal.

In yet another aspect, the invention provides a cell generated by a method according to the invention.

In yet another aspect, the invention provides a MVA reproduced using a cell according to the invention.

In yet another aspect, the invention provides the use of poxvirus host range genes CP77 and K1L for rendering a cell, preferably a cell of a continuous cell line, capable of expressing CP77 and K1L genes.

In yet another aspect, the invention provides the use of poxvirus host range genes CP77 and K1L for rendering a cell, preferably a cell of a continuous cell line, capable of allowing MVA replication in said cell.

In one embodiment of the use, a nucleotide sequence encoding an amino acid sequence of SEQ ID NO: 1 and a nucleotide sequence encoding an amino acid sequence of SEQ ID NO: 3 is used for rendering the cell capable of expressing CP77 and K1L genes.

In one embodiment of the use, a nucleotide sequence of SEQ ID NO: 2 and a nucleotide sequence of SEQ ID NO: 4 is used for rendering the cell capable of expressing CP77 and K1L genes.

In yet another aspect, the invention provides the use of poxvirus host range genes CP77, K1L and SPI-1 for rendering a cell, preferably a cell of a continuous cell line, capable of expressing CP77, K1L and SPI-1 genes.

In yet another aspect, the invention provides the use of poxvirus host range genes CP77, K1L and SPI-1 for rendering a cell, preferably a cell of a continuous cell line, capable of allowing MVA replication in said cell.

In one embodiment of the use, a nucleotide sequence encoding an amino acid sequence of SEQ ID NO: 1, a nucleotide sequence encoding an amino acid sequence of SEQ ID NO: 3, and a nucleotide sequence encoding an amino acid of SEQ ID NO: 9 is used for rendering the cell capable of expressing CP77, K1L and SPI-1 genes.

In one embodiment of the use, a nucleotide sequence of SEQ ID NO: 2, a nucleotide sequence of SEQ ID NO: 4 and a nucleotide sequence of SEQ ID NO: 10 is used for rendering the cell capable of expressing CP77, K1L and SPI-1 genes.

In yet another aspect, the invention provides a MVA expressing poxvirus host range genes CP77 and K1L.

In one embodiment of the MVA, the MVA comprises a nucleotide sequence encoding an amino acid sequence of SEQ ID NO: 1 and a nucleotide sequence encoding an amino acid sequence of SEQ ID NO: 3.

In one embodiment of the MVA, the MVA comprises a nucleotide sequence of SEQ ID NO: 2 and a nucleotide sequence of SEQ ID NO: 4.

In yet another aspect, the invention provides a MVA expressing poxvirus host range genes mRFP1-CP77 and K1L-V5.

In one embodiment of the MVA, the MVA comprises a mRFP1-CP77 fusion gene, preferably wherein a CP77 gene is fused via a flexible linker to the C-terminus of RFP.

In one embodiment of the MVA, the MVA comprises a nucleotide sequence encoding an amino acid sequence of SEQ ID NO: 5 and a nucleotide sequence encoding an amino acid sequence of SEQ ID NO: 7.

In one embodiment of the MVA, the MVA comprises a nucleotide sequence of SEQ ID NO: 6 and a nucleotide sequence of SEQ ID NO: 8.

In yet another aspect, the invention provides a MVA expressing poxvirus host range genes CP77, K1L and SPI-1.

In one embodiment of the MVA, the MVA comprises a nucleotide sequence encoding an amino acid sequence of SEQ ID NO: 5, a nucleotide sequence encoding an amino acid sequence of SEQ ID NO: 7, and a nucleotide sequence encoding an amino acid sequence of SEQ ID NO: 9.

In one embodiment of the MVA, the MVA comprises a nucleotide sequence of SEQ ID NO: 6, a nucleotide sequence of SEQ ID NO: 8, and a nucleotide sequence of SEQ ID NO: 10.

In one embodiment of the MVA, a host range gene is operably linked to a promoter selected from the group consisting of PrH5m, Pr1328 and Pr13.5.

In one embodiment of the MVA, the MVA comprises mRFP1-CP77 or CP77 gene operably linked to PrH5m promoter.

In one embodiment of the MVA, the MVA comprises K1L-V5 or K1L gene operably linked to Pr1328 promoter.

In one embodiment, the MVA comprises a SPI-1 host range gene operably linked to Pr13.5 promoter.

In one embodiment of the MVA, the insertion site of mRFP1-CP77 or CP77 gene is the intergenic region between ORF MVA44L and MVA45L (IGR 44/45).

In one embodiment of the MVA, the insertion site of K1L-V5 or K1L gene is between ORF MVA88R and MVA89L.

In one embodiment of the MVA, the insertion site of SPI-1 host range gene is between ORF MVA88R and MVA89L.

In one embodiment of the MVA, the MVA comprises an expression cassette comprising mRFP1-CP77 or CP77 gene operably linked to PrH5m promoter, which expression cassette is inserted into insertion site MVA44L and MVA45L (IGR 44/45).

In one embodiment of the MVA, the MVA comprises an expression cassette comprising K1L-V5 or K1L gene operably linked to Pr1328 promoter, which expression cassette is inserted into insertion site MVA88R and MVA89L.

In one embodiment of the MVA, the MVA comprises an expression cassette comprising SPI-1 host range gene operably linked to Pr13.5 promoter, which expression cassette is inserted into insertion site MVA88R and MVA89L.

Embodiments relating to MVA

In one embodiment, the recombinant MVA is generated from an MVA selected from the group consisting of MVA-572, MVA-575, MVA-1721, NIH clone 1 and MVA-BN, preferably from MVA-BN or a derivative thereof

MVA-572 has been deposited as ECACC V94012707 on 27 Jan. 1994; MVA-575 has been deposited as ECACC V00120707 on 7 Dec. 2000; MVA-1721 is referenced in Suter et al. Vaccine 2009, 27:7442-7450; NIH clone 1 has been deposited as ATCC® PTA-5095 on 27 Mar. 2003; and MVA-BN has been deposited at the European Collection of Cell Cultures (ECACC) under number V00083008 on 30 Aug. 2000.

In one embodiment, the recombinant MVA is a recombinant MVA-BN or a recombinant MVA-BN derivative.

### FURTHER DESCRIPTION

### Modified Vaccinia Virus Ankara (MVA)

In the past, MVA was generated by 516 serial passages on chicken embryo fibroblasts of the Ankara strain of vaccinia virus (CVA) (for review see Mayr et al. 1975). This virus was renamed from CVA to MVA at passage 516 to account for its substantially altered properties. MVA was subjected to further passages up to a passage number of over 570. As a consequence of these long-term passages, the genome of the resulting MVA virus had about 31 kilobases of its genomic sequence deleted and, therefore, was described as highly host cell restricted for replication to avian cells (Meyer et al. 1991). It was shown in a variety of animal models that the resulting MVA was significantly avirulent compared to the fully replication competent starting material (Mayr and Danner 1978).

An MVA useful in the practice of the present invention includes MVA-572 (deposited as ECACC V94012707 on 27 Jan. 1994); MVA-575 (deposited as ECACC V00120707 on 7 Dec. 2000), MVA-1721 (referenced in Suter et al. 2009), NIH clone 1 (deposited as ATCC® PTA-5095 on 27 Mar. 2003) and MVA-BN (deposited at the European Collection of Cell Cultures (ECACC) under number V00083008 on 30 Aug. 2000).

More preferably the MVA used in accordance with the present invention includes MVA-BN and MVA-BN derivatives. MVA-BN has been described in WO 02/042480. “MVA-BN derivatives” refer to any virus exhibiting essentially the same replication characteristics as MVA-BN, as described herein, but exhibiting differences in one or more parts of their genomes.

MVA-BN, as well as MVA-BN derivatives, is replication incompetent, meaning a failure to reproductively replicate in vivo and in vitro. More specifically in vitro, MVA-BN or MVA-BN derivatives have been described as being capable of reproductive replication in chicken embryo fibroblasts (CEF), but not capable of reproductive replication in the human keratinocyte cell line HaCat (Boukamp et al 1988), the human bone osteosarcoma cell line 143B (ECACC Deposit No. 91112502), the human embryo kidney cell line 293 (ECACC Deposit No. 85120602), and the human cervix adenocarcinoma cell line HeLa (ATCC Deposit No. CCL-2). Additionally, MVA-BN or MVA-BN derivatives have a virus amplification ratio at least two-fold less, more preferably three-fold less than MVA-575 in Hela cells and HaCaT cell lines. Tests and assay for these properties of MVA-BN and MVA-BN derivatives are described in WO 02/42480 and WO 03/048184.

The term “not capable of reproductive replication” in human cell lines in vitro as described above is, for example, described in WO 02/42480, which also teaches how to obtain MVA having the desired properties as mentioned above. The term applies to a virus that has a virus amplification ratio in vitro at 4 days after infection of less than 1 using the assays described in WO 02/42480 or U.S. Pat. No. 6,761,893.

### Exemplary Generation of a Recombinant MVA Virus

For the generation of a recombinant MVA as described herein, different methods may be applicable. The DNA sequence to be inserted into the virus can be placed into an E. coli plasmid construct into which DNA homologous to a section of DNA of the poxvirus has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of poxvirus DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA viral DNA in the plasmid and the viral genome, respectively, can generate an MVA modified by the presence of foreign DNA sequences, i.e., nucleotides sequences encoding SARS-COV-2 antigens.

According to a preferred embodiment, a cell of a suitable cell culture as, e.g., CEF cells, can be infected with a MVA virus. The infected cell can be, subsequently, transfected with a first plasmid vector comprising a foreign or heterologous gene or genes, such as one or more of the nucleic acids provided herein, preferably under the transcriptional control of a poxvirus expression control element. As explained above, the plasmid vector also comprises sequences capable of directing the insertion of the exogenous sequence into a selected part of the MVA viral genome. Optionally, the plasmid vector also contains a cassette comprising a marker and/or selection gene operably linked to a poxvirus promoter. The use of selection or marker cassettes simplifies the identification and isolation of the generated recombinant MVA. However, a recombinant poxvirus can also be identified by PCR technology. Subsequently, a further cell can be infected with the recombinant MVA obtained as described above and transfected with a second vector comprising a second foreign or heterologous gene or genes. In case, this gene shall be introduced into a different insertion site of the poxvirus genome, the second vector also differs in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection. There are ample of other techniques known to generate recombinant MVA.

The practice of the invention will employ, if not otherwise specified, conventional techniques of immunology, molecular biology, microbiology, cell biology, and recombinant technology, which are all within the skill of the art. See e.g. Sambrook, Fritsch and Maniatis, Molecular Cloning: A Laboratory Manual, 2nd edition, 1989; Current Protocols in Molecular Biology, Ausubel F M, et al., eds, 1987; the series Methods in Enzymology (Academic Press, Inc.); PCR2: A Practical Approach, MacPherson M J, Hams B D, Taylor G R, eds, 1995; Antibodies: A Laboratory Manual, Harlow and Lane, eds, 1988.

### EXAMPLES

The following examples serve to further illustrate the disclosure. They should not be construed as limiting the invention the scope of which is determined by the appended claims.

### Example 1: Preparation of Recombinant Vaccinia Virus

### 1.1 Recombinant MVA and CVA

MVA recombinants encoding poxvirus host ranges genes intrinsically absent in MVA (see Table 2 below) and a modified MVA referred to as “wildtype” were derived from MVA-BN®. CVA as the MVA parent strain was used for comparison: A CVA recombinant encoding CP77 (intrinsically absent in CVA) and “wildtype” CVA.

MVA and CVA were reconstituted from bacterial artificial chromosome (BAC) clones constructed from MVA-BN® and characterized as previously described (12). Both MVA and CVA BAC clones contained a bi-cistronic expression cassette under the control of the strong synthetic early/late pS promoter, which cassette encoded for a neomycin-phosphotransferase selection marker (NPT II).

The clones furthermore contained an internal ribosome entry site (IRES) and the gene for enhanced green fluorescent protein (EGFP) reporter protein.

Recombinant MVA and CVA were reconstituted from BAC clones encoding EGFP which clones had been modified to contain the host range genes of interest. MVA and CVA reconstituted from unmodified BAC clones encoding EGFP were referred to as “wildtype”, i.e., “MVA-wt” and “CVA-wt”, respectively. During the generation of virus stocks, no significant difference was observed between titers obtained from recombinant MVA and MVA-wt.

### 1.2 Cell Culture

Primary CEF cells were prepared from 11-day-old embryonated chicken eggs (VALO BioMedia GmbH) and cultured in VP-SFM medium (Gibco).

CHO cells were obtained from ATCC (CCL-61, CHO-K1) and cultured in Nutrient Mixture F-12 Ham medium (Sigma-Aldrich) supplemented with 10% fetal calf serum (FCS).

BHK-21 cells (obtained from ATCC) were grown in Dulbecco's modified Eagle medium (DMEM) (Gibco/Thermos Fisher Scientific) supplemented with 10% FCS (Pan Biotech).

### 1.3 Generation of Recombinant CVA

Recombinant CVA encoding CP77 (“CVA-C”) was prepared by homologous recombination. For ease of detection, the CP77 gene was fused to the C-terminus of mono red fluorescence protein (mRFP1) via a flexible linker.

A plasmid pMISC564 containing the flanks for homologous recombination between MVA open reading frame 44 and 45, and the mRFP1-CP77 fusion gene driven by the PrH5m promoter was synthesized by Invitrogen GeneArt (Thermo Fisher Scientific). pBN564 was linearized with SacI and NheI and transfected into monolayers of CHO cells infected with CVA-wt using Fugene HD (Roche Diagnostics). At 48 hours after transfection, cells and supernatant were harvested and sonicated with a cup sonicator. To eliminate CVA-wt from the virus preparation, recombinant CVA containing the mRFP1-CP77 fusion gene was selected by passaging in CHO cells. After four passages in CHO cells, total DNA was extracted from infected cells with NucleoSpin Blood Mini kit (Macherey-Nagel) and screened by PCR to verify the absence of unwanted CVA-wt DNA and the presence of the desired recombinant CVA-C.

### 1.4 Generation of Recombinant MVA

MVA recombinants encoding the host range genes of interest are summarized in Table 2 below.

TABLE 2
Recombinant MVA and host range genes inserted.

| Recombinant | |
|---|---|
| MVA | Host range genes |
| MVA-C | mRFP1-CP77 |
| MVA-CK | mRFP1-CP77 + K1L-V5 |
| MVA-CKS | mRFP1-CP77 + K1L-V5 + SPI-1 |
| MVA-CKS-C9 | mRFP1-CP77 + K1L-V5 + SPI-1 + C9L |
| MVA-KS | K1L-V5 + SPI-1 |

The design of MVA recombinants, i.e., expression cassettes with host range gene and promoter as well as the cassettes' insertion sites, is illustrated in FIG. 1.

### 1.4.1 Preparation of MVA-BAC Clones

MVA recombinants were prepared using the BAC-A Red recombination technology.

All BAC clones were propagated in E. coli strain MDS42 containing plasmid pKD46. BAC DNA was extracted with NucleoBond® Xtra BAC kit (Macherey-Nagel). The host range genes of interest driven by selected poxviral promoters were inserted into the MVA BAC clone using the A Red recombination system as described previously (12, 31, 32). Briefly, a counter selectable rpsL/neo cassette bearing a positive and a negative selection marker flanked by homology arms was generated by PCR. The rpsL/neo cassette was electroporated into E. coli strain MDS42 carrying MVA BAC and plasmid pKD46 (33). Plasmid pKD46 encoding proteins for A Red recombination was provided by B. L. Wanner [27]. With the A Red recombination proteins expressed from pKD46, the rpsL/neo cassette was inserted into the corresponding insertion site directed by the homologous sequences in the PCR fragment. In a second recombination step, the rpsl/neo cassette was replaced with a fragment encoding the host range gene(s) (in plasmid pMISC564 or pMISC576). Plasmid pMISC564 containing the flanks for homologous recombination and the mRFP1-CP77 gene under the control of the PrH5m promoter was synthesized by GeneArt (ThermoFisher Scientific). To monitor the spread of recombinant viruses in cell substrates, CP77 was fused to the C-terminus of mRFP1 with a glycine-serine (GS) linker like the GFP-CP77 fusion protein that has been described previously (34). Plasmid pMISC576 containing the flanks for homologous recombination, the K1L-V5 gene under the control of the Pr1328 promoter, and the SPI-1 gene driven by the Pr13.5 promoter was synthesized by GeneArt (ThermoFisher Scientific). Because no antibody against K1L is available, a V5 tag was added to the C-terminus of the K1L gene by a GS linker for detecting the K1L protein. Because the C9L is truncated in MVA and CVA, to generate the MVA-CKS-C9, the C9L gene was repaired in MVA-CKS in situ. Briefly, the truncated C9L gene in the MVA-CKS BAC clone was replaced with the rpsL/neo cassette. In a second recombination step, the rpsL/neo cassette was replaced with C9L DNA sequence which was amplified with PCR by using the VACV-WR genomic DNA as template (VACV-WR was from ATCC). Successful recombination at each step was confirmed by PCR and sequencing.

### 1.4.2 Reconstitution of Infectious Recombinant MVA

Infectious MVA recombinants were reconstituted from the respectively constructed MVA-BAC clones as previously described (12). Briefly, BHK-21 cells seeded in 6-well plates were transfected with 3 μg of MVA-BAC DNA using Fugene HD (Promega) and 60 min later infected with the helper virus Shope Fibroma Virus (SFV) (obtained from ATCC). The cells were monitored for EGFP expression and harvested 3 days after transfection. The cell lysate was used to infect CEF cells, and three cell passages were performed to remove the helper virus SFV, which cannot propagate in CEF cells. Total DNA was extracted from infected cells and used for detection of residual (contaminating) SFV by means of PCR.

### 1.5 Analysis of Host Range Gene Expression

The expression of genes inserted into CVA or MVA was analyzed using reverse transcription-PCR (RT-PCR). For RNA extraction and analysis, CEF cells in 6-well plates were infected with CVA-wt, CVA-C, MVA-wt, or MVA recombinants MVA-C, MVA-CK, MVA-CKS, MVA-CKS-C9, or MVA-KS.

After 48 hours of infection, the supernatants were removed, and cells were scraped and resuspended into 350 μl RTL lysis buffer. Cell lysates were homogenized using QIAshredder columns (Qiagen), and genomic DNA was removed using gDNA eliminator columns (Qiagen). RNA in the flow-through from genomic DNA eliminator columns was purified with the RNeasy

Plus mini kit (Qiagen) according to the manufacturer's instructions. RNA was eluted from the RNeasy spin columns with 50 μl RNase-free water. The remaining viral and cellular DNA in the purified samples was digested with Turbo DNase (Ambion/Life Technologies), followed by DNase inactivation with EDTA (Sigma-Aldrich) to a final concentration of 15 mM. Reverse transcription of 3 μl samples of isolated RNA was performed using the OneTaq RT-PCR kit (NEB) according to the manufacturer's instructions. Samples without reverse transcriptase (RT) were set up as negative controls. A total of 2 μl of RT reaction mixture was used to detect the transcription of the different host range genes with OneTaq Hot Start 2X Master Mix (NEB) according to the manufacturer's instructions. Detection of EGFP transcripts was performed as positive control. Primers used for detection of the genes and the size of the expected PCR products are listed in Table 3.

TABLE 3
Primers for RT-PCR detection of mRNA and expected product size

| | Primers | | Size |
|---|---|---|---|
| Gene | Forward primer | Reverse primer | (bp) |
| EGFP | CAGCTCGTCCATGCCGAGAG | TGAAGTTCATCTGCACCACC | 578 |
| CP77 | TGAACCCTGAGGTGGTCAAGTGC | CGATCAGGATGTGCTGGAGCAC | 390 |
| K1L | CGATGTGCACGGACACAGC | GCTGGTCATGTAGTCCAGCAGC | 478 |
| SPI-1 | CGATCAGGATGTGCTGGAGCAC | GTACACCTTGGTCCGGTACACC | 465 |
| C9L | CATGCTACATGTGTACTTATAAT | GCCGTATATTGATGATATAAACAA | 379 |
| | CGAC | AATAG | |

As shown in FIG. 2, transcripts from all inserted host range genes (i.e., mRFP1-CP77 fusion gene and the genes of K1L-V5, SPI-1, and C9L) were detected by RT-PCR and thus expressed by the MVA and CVA recombinants. The detection of EGFP transcripts in samples treated with reverse transcriptase served as a control. In the case of C9L, only a truncated defective transcript was detected which was possible because the primers targeted the residual sequence of the defective C9L transcript (7).

### Example 2: Replication of Recombinant Vaccinia Virus in CHO Cells

The effect of the inserted host range genes on replication properties of the MVA recombinants was investigated in CHO cells as an example of a non-permissive mammalian cell line.

### 2.1 Cell Culture

CHO cell culture and CEF cell preparation was as briefly described above (see Example 1.1).

### 2.2 Viral Replication in CHO Cells

For analysis of virus replication and spread, confluent CHO cell monolayers in 6-well culture plates were infected at 0.1 TCID₅₀ per cell using 1×10⁵ TCID₅₀ in 500 μl of DMEM without FCS. After 60 min at 37° C., cells were washed once with DMEM and further incubated with 2 ml of DMEM containing 2% FCS. For infection, CHO cells were incubated at 37° C. with 2 ml of F-12 Ham medium containing 2% FCS. Virus spread was determined by detecting EGFP using fluorescence microscopy at 72 hours post infection.

As shown in FIG. 3, no detectable expression of the viral reporter genes EGFP and mRFP1 were observed in CHO cells infected with CVA-wt. In contrast, CHO cells infected with CVA-C showed widespread EGFP and mRFP1 signals as well as notable cytopathic effects (CPE), the latter being indicative of a fully permissive cell line. Apparently, the mRP1-CP77 fusion gene in CVA-C expressed a protein fully functional with respect to supporting MVA replication in CHO cells.

As furthermore shown in FIG. 3, CHO cells were not permissive for MVA-wt. In contrast, mRFP1 and EGFP signals as well as CPE were particularly observed for MVA-CKS and to a lower extent for MVA-C and MVA-CK. Replication of MVA-KS in CHO cells, however, was not better than that in MVAS-wt.

### 2.3 Replication Capacity of MVA Recombinants in CHO Cells

Replication capacity, a measure for how quickly a virus replicates, was investigated.

CHO cells were infected as described above (see Example 2.2). Cells and supernatants were harvested, sonicated to release virus and titrated on CEF cells according to the TCID₅₀ method (35). Statistical analyses were performed using GraphPad PRISM (GraphPad Software, San Diego, USA).

As shown in FIG. 4, all MVA recombinants expressing CP77 gene replicated in CHO cells, and they did it in the following order: MVA-CKS=MVA-CKS-9>MVA-CK>MVA-C.

MVA expressing only CP77 (MVA-C) replicated in CHO cells with titers at least 1 log higher than those produced by MVA-wt. Titers produced in CHO cells infected with MVA-CKS were nearly 3 logs higher than with MVA-C. The strongest step of improvement in titer, by about two orders of magnitude, was observed with MVA-CKS as compared to MVA-CK.

Thus, the insertion of poxvirus host range genes into MVA, i.e., mRFP1-CP77 (MVA-C), followed by the addition of K1L-V5 (MVA-CK) and the subsequent addition of SPI-1 (MVA-CKS), resulted in a significant stepwise improvement in the replication capacity of MVA in CHO cells.

Interestingly, insertion of the C9L gene in addition to CP77, K1L and SPI-1 genes (MVA-CKS-C9) did not result in a further increase in viral titer as compared to that of MVA-CKA. The virus titer of MVA-KS was even lower than that measured for MVA-wt.

Notably, the titers obtained for MVA-CKS in CHO cells were equivalent to those routinely obtained for MVA-wt in primary CEF cells.

In conclusion, CP77 gene is required for MVA replication in CHO cells, but K1-L and SPI-1 in combination with CP77 makes the MVA recombinant replicate in the order known for replication of native MVA in CEF cells.

### 2.4 Growth of MVA Recombinants in CHO Cells

The growth kinetics of MVA-CKS in CHO cells was also analyzed.

Briefly, CHO cells were infected with MVA-wt or MVA-CKS and cultured as described above (see Example 2.2). MVA was harvested at different times and titrated on CEF cells as described above (see Example 2.3).

As shown in FIG. 5, MVA-CKS efficiently replicated in CHO cells within 24 hours post infection with reaching a maximum virus titer at 48 to 72 hours. FIG. 4 also demonstrates that MVA-wt cannot replicate in CHO cells.

### Example 3: Replication of Recombinant Vaccinia Virus in Further Cells

### 3.1 Cell Culture

CEF cells were prepared as described above (see Example 1.2).

All cell lines were obtained from American Type Culture Collection (ATCC) or European Collection of Authenticated Cell Cultures (ECACC).

CHO cells were cultured as briefly described above (see Example 1.2).

Cell lines other than CHO cells were grown in Dulbecco's modified Eagle medium (DMEM) (Gibco/Thermos Fisher Scientific) supplemented with 10% fetal calf serum (FCS) (Pan Biotech).

### 3.2 Viral Replication in HEK293, RK13 and Chicken Cells

Viral replication in HEK293 and RK13 cells was analyzed by fluorescence microscopy as described above (see Example 2.2).

As shown in FIG. 6, human embryonic kidney HEK293 cells were not permissive for MVA-wt, while they were permissive for CVA-wt. However, none of the MVA recombinants replicated in HEK293 cells.

In contrast, as shown in FIG. 7, RK13 cells which were permissive for CVA-wt and all MVA recombinants, but not for MVA-wt. Because the K1L is a VACV host range gene that is known to restore replication of MVA in RK13 cells (21) and the host range function of K1L can be complemented by CP77 in RK13 cells (17), RK13 cells were used here as a control cell substrate to verify the host range function of K1L-V5 and mRFP1-CP77 fusion proteins. The results that RK13 cells are permissive for MVA-C and MVA-KS confirmed the host range function of K1L-V5 and mRFP1-CP77.

Furthermore, viral replication of MVA-CKS and MVA-wt in terms of virus titer produced in cells derived from chicken was analyzed as described above (see Example 2.3).

Primary CEF cells and cells of the chicken fibroblast DF-1 cell line were permissive both for MVA-wt and MVA-CKS and no significant difference between MVA-CKS and MVA-wt was found in their ability to infect CEF and DF-1 cells.

### Example 4: Preparation of CHO Cells Permissive for MVA

Based on the findings that MVA-CKS replicated in CHO cells (see Example 2 above), generation of CHO cells stably expressing poxvirus host range genes CP77, K1L and SPI-1 was considered. Methods of generating a CHO cell line that stably expresses transgenes are available (e.g., 18).

### 4.1 Cloning of Plasmid Containing CP77, K1L, SPI-1, and NPT II Genes

A plasmid containing the three host range genes CP77, K1L and SPI-1 driven by different promoters (CMV promoter, β-globin promoter, and β-actin promoter, respectively) for gene expression in mammalian cells was prepared. The plasmid furthermore contained an NPT II-IRES-EGFP cassette driven by SV40 promoter as selection marker and reporter gene.

### 4.2 Transfection of CHO Cells and Clone Analysis

CHO cells (ATCC CCL-61) seeded in a 6-well plate with F-12 Ham medium supplemented with 10% FCS were transfected with 1 μg linearized plasmid by using FuGENE® HD transfection reagent (Promega) according to the manufacturer's instructions. After 24 h, medium was replaced with fresh medium containing antibiotic selection (G418). The cell line was established by single-cell sorting, followed by further expansion with antibiotic selection. Clones were analyzed by detecting the transcription of the transgenes by RT-PCR. Positive clones were further tested by infecting with MVA. Viruses were harvested at 48 h after infection and titrated.

Final remark: Several documents are cited throughout the text of this specification. Each of the documents cited herein (including all patents, patent applications, scientific publications, manufacturer's specifications, instructions, etc.) are hereby incorporated by reference in their entirety. To the extent, the material incorporated by reference contradicts or is inconsistent with this specification, the specification will supersede any such material. Nothing herein is to be construed as an admission that the invention is not entitled to antedate such disclosure by virtue of prior invention.

### REFERENCES

1. Stickl H, Hochstein-Mintzel V, Mayr A, Huber H C, Schäfer H, Holzner A. 1974. [MVA vaccination against smallpox: clinical tests with an attenuated live vaccinia virus strain (MVA) (author's transl)]. Dtsch Med Wochenschr 99:2386-92.
2. Kennedy J S, Greenberg R N. 2009. IMVAMUNE: modified vaccinia Ankara strain as an attenuated smallpox vaccine. Expert Rev Vaccines 8:13-24.
3. Volz A, Sutter G. 2017. Modified Vaccinia Virus Ankara: History, Value in Basic Research, and Current Perspectives for Vaccine Development. Adv Virus Res 97:187-243.
4. Gomez C E, Perdiguero B, Garcia-Arriaza J, Esteban M. 2013. Clinical applications of attenuated MVA poxvirus strain. Expert Rev Vaccines 12:1395-416.
5. Sutter G, Moss B. 1992. Nonreplicating vaccinia vector efficiently expresses recombinant genes. Proc Natl Acad Sci USA 89:10847-51.
6. Mayr A, Hochstein-Mintzel V, Stickl H. 1975. Abstammung, Eigenschaften und Verwendung des attenuierten Vaccinia-Stammes MVA. Infection 3:6-14.
7. Meisinger-Henschel C, Schmidt M, Lukassen S, Linke B, Krause L, Konietzny S, Goesmann A, Howley P, Chaplin P, Suter M, Hausmann J. 2007. Genomic sequence of chorioallantois vaccinia virus Ankara, the ancestor of modified vaccinia virus Ankara. J Gen Virol 88:3249-59.
8. Antoine G, Scheiflinger F, Dorner F, Falkner F G. 1998. The complete genomic sequence of the modified vaccinia Ankara strain: comparison with other orthopoxviruses. Virology 244:365-96.
9. Meyer H, Sutter G, Mayr A. 1991. Mapping of deletions in the genome of the highly attenuated vaccinia virus MVA and their influence on virulence. J Gen Virol 72 (Pt 5): 1031-8.
10. Carroll M W, Moss B. 1997. Host range and cytopathogenicity of the highly attenuated MVA strain of vaccinia virus: propagation and generation of recombinant viruses in a nonhuman mammalian cell line. Virology 238:198-211.
11. Blanchard T J, Alcami A, Andrea P, Smith G L. 1998. Modified vaccinia virus Ankara undergoes limited replication in human cells and lacks several immunomodulatory proteins: implications for use as a human vaccine. J Gen Virol 79 (Pt 5): 1159-67.
12. Meisinger-Henschel C, Spath M, Lukassen S, Wolferstatter M, Kachelriess H, Baur K, Dirmeier U, Wagner M, Chaplin P, Suter M, Hausmann J. 2010. Introduction of the six major genomic deletions of modified vaccinia virus Ankara (MVA) into the parental vaccinia virus is not sufficient to reproduce an MVA-like phenotype in cell culture and in mice. J Virol 84:9907-19.
13. Spehner D, Gillard S, Drillien R, Kirn A. 1988. A cowpox virus gene required for multiplication in Chinese hamster ovary cells. J Virol 62:1297-304.
14. Perkus M E, Goebel S J, Davis S W, Johnson G P, Limbach K, Norton E K, Paoletti E. 1990. Vaccinia virus host range genes. Virology 179:276-286.
15. Ramsey-Ewing A, Moss B. 1995. Restriction of vaccinia virus replication in CHO cells occurs at the stage of viral intermediate protein synthesis. Virology 206:984-93.
16. Shchelkunov S N, Safronov P F, Totmenin A V, Petrov N A, Ryazankina Ol, Gutorov V V, Kotwal G J. 1998. The genomic sequence analysis of the left and right species-specific terminal region of a cowpox virus strain reveals unique sequences and a cluster of intact ORFs for immunomodulatory and host range proteins. Virology 243:432-60.
17. Ramsey-Ewing A L, Moss B. 1996. Complementation of a vaccinia virus host-range K1L gene deletion by the nonhomologous CP77 gene. Virology 222:75-86.
18. Eldi P, Cooper T H, Liu L, Prow N A, Diener K R, Howley P M, Suhrbier A, Hayball J D. 2017. Production of a Chikungunya Vaccine Using a CHO Cell and Attenuated Viral-Based Platform Technology. Mol Ther 25:2332-2344.
19. Lynch H E, Ray C A, Oie K L, Pollara J J, Petty I T, Sadler A J, Williams B R, Pickup D J. 2009. Modified vaccinia virus Ankara can activate NF-kappaB transcription factors through a double-stranded RNA-activated protein kinase (PKR)-dependent pathway during the early phase of virus replication. Virology 391:177-86.
20. McFadden G. 2005. Poxvirus tropism. Nat Rev Microbiol 3:201-13.
21. Sutter G, Ramsey-Ewing A, Rosales R, Moss B. 1994. Stable expression of the vaccinia virus K1L gene in rabbit cells complements the host range defect of a vaccinia virus mutant. J Virol 68:4109-16.
22. Ali A N, Turner P C, Brooks M A, Moyer R W. 1994. The SPI-1 gene of rabbitpox virus determines host range and is required for hemorrhagic pock formation. Virology 202:305-14.
23. Liu R, Moss B. 2018. Vaccinia Virus C9 Ankyrin Repeat/F-Box Protein Is a Newly Identified Antagonist of the Type I Interferon-Induced Antiviral State. J Virol 92.
24. Werden S J, Rahman M M, McFadden G. 2008. Poxvirus host range genes. Adv Virus Res 71:135-71.
25. Bratke K A, McLysaght A, Rothenburg S. 2013. A survey of host range genes in poxvirus genomes. Infection, Genetics and Evolution.
26. Shisler J L, Jin X L. 2004. The vaccinia virus K1L gene product inhibits host N F-kappaB activation by preventing IkappaBalpha degradation. J Virol 78:3553-60.
27. Brooks M A, Ali A N, Turner P C, Moyer R W. 1995. A rabbitpox virus serpin gene controls host range by inhibiting apoptosis in restrictive cells. J Virol 69:7688-98.
28. Shisler J L, Isaacs S N, Moss B. 1999. Vaccinia virus serpin-1 deletion mutant exhibits a host range defect characterized by low levels of intermediate and late mRNAs. Virology 262:298-311.
29. Liu R, Mendez-Rios J D, Peng C, Xiao W, Weisberg A S, Wyatt L S, Moss B. 2019. SPI-1 is a missing host-range factor required for replication of the attenuated modified vaccinia Ankara (MVA) vaccine vector in human cells. PLOS Pathog 15: e1007710.
30. Peng C, Moss B. 2020. Repair of a previously uncharacterized second host-range gene contributes to full replication of modified vaccinia virus Ankara (MVA) in human cells. Proc Natl Acad Sci USA 117:3759-3767.
31. Wang S, Zhao Y, Leiby M, Zhu J. 2009. A new positive/negative selection scheme for precise BAC recombineering. Mol Biotechnol 42:110-6.
32. Reyrat J M, Pelicic V, Gicquel B, Rappuoli R. 1998. Counterselectable markers: untapped tools for bacterial genetics and pathogenesis. Infect Immun 66:4011-7.
33. Datsenko K A, Wanner B L. 2000. One-step inactivation of chromosomal genes in Escherichia coli K-12 using PCR products. Proc Natl Acad Sci USA 97:6640-5.
34 Hsiao J C, Chao C C, Young M J, Chang Y T, Cho E C, Chang W. 2006. A poxvirus host range protein, CP77, binds to a cellular protein, HMG20A, and regulates its dissociation from the vaccinia virus genome in CHO-K1 cells. J Virol 80:7714-28.
35. Staib C, Drexler I, Sutter G. 2004. Construction and isolation of recombinant MVA. Methods Mol Biol 269:77-100.

We claim:

1. A cell of a continuous cell line, the cell being genetically modified to express poxvirus host range genes CP77 and K1L.

2. The cell of claim 1, the genome of which comprises poxvirus host range genes CP77 and K1L, preferably comprising a nucleotide sequence encoding an amino acid sequence of SEQ ID NO: 1 and a nucleotide sequence encoding an amino acid sequence of SEQ ID NO: 3.

3. The cell of claim 1, the cell being genetically modified to express poxvirus host range genes CP77, KIL and SPI-1.

4. The cell of claim 3, the genome of which comprises poxvirus host range genes CP77, KIL and SPI-1, preferably comprising a nucleotide sequence encoding an amino acid sequence of SEQ ID NO: 1, a nucleotide sequence encoding an amino acid sequence of SEQ ID NO: 3, and a nucleotide sequence encoding an amino acid sequence of SEQ ID NO: 9.

5. The cell of claim 1 which is a cell of a non-human mammalian cell line, preferably is a Chinese hamster ovary (CHO) cell.

6. The cell of claim 1 which is infected with Modified Vaccinia Virus Ankara (MVA).

7. A Modified Vaccinia Virus Ankara (MVA) produced by the cell of claim 6.

8. A vaccine comprising the Modified Vaccinia Virus Ankara (MVA) of claim 7.

9. A vaccine comprising Modified Vaccinia Virus Ankara (MVA) produced by the cell of claim 1.

10. A method for generating a cell of claim 1, comprising the following steps:
(a) preparing a nucleic acid suitable for gene transfer into a cell of a continuous cell line, the nucleic acid comprising:
(i) poxvirus host range gene CP77 operably linked to a promoter; or
(ii) poxvirus host range gene KIL operably linked to a promoter; or
(iii) poxvirus host range genes CP77 and K1L, each operably linked to a promoter;
(b) introducing nucleic acids (i) and (ii) obtained in step (a), or nucleic acid (iii) obtained in step (a), into the cell; and
(c) selecting a cell population or clone expressing poxvirus host range genes CP77 and K1L.

11. A method for generating the cell of claim 3, comprising the following steps:
(a) preparing a nucleic acid suitable for gene transfer into a cell of a continuous cell line, the nucleic acid comprising:
(i) poxvirus host range gene CP77 operably linked to a promoter; or
(ii) poxvirus host range gene KIL operably linked to a promoter; or
(iii) poxvirus host range gene SPI-1 operably linked to a promoter; or
(iv) poxvirus host range genes CP77, K1L and SPI-1, each operably linked to a promoter;
(b) introducing nucleic acids (i), (ii) and (iii) obtained in step (a), or nucleic acid (iv) obtained in step (a), into the cell; and
(c) selecting a cell population or clone expressing poxvirus host range genes CP77, KIL and SPI-1.

12. A Modified Vaccinia Virus Ankara (MVA) produced by the cell of claim 3.

13. The method of claim 10, wherein said nucleic acid comprising host range gene CP77 encodes the amino acid sequence of SEQ ID NO: 1 and said nucleic acid comprising host range gene KIL encodes the amino acid sequence of SEQ ID NO: 3.

14. The method of claim 11, wherein said nucleic acid comprising host range gene SPI-1 encodes the amino acid sequence of SEQ ID NO: 9.

15. The method of claim 13, wherein the cell is a cell of a non-human mammalian cell line, preferably is a Chinese hamster ovary (CHO) cell.

16. A Modified Vaccinia Virus Ankara (MVA) comprising poxvirus host range genes CP77 and K1L, preferably comprising a nucleotide sequence encoding an amino acid sequence of SEQ ID NO: 1 and a nucleotide sequence encoding an amino acid sequence of SEQ ID NO: 3.

17. The MVA of claim 16, further comprising poxvirus host range gene SPI-1, preferably further comprising a nucleotide sequence encoding an amino acid sequence of SEQ ID NO: 9.
